SEQUENCE LISTING (2 sequences)

SEQ ID NO: 1 - DNA, 1563 bp, Mus sp.
ggcaacagacacctttaccaacttggttcttcttcttcttcttcttcttcttcttcttcttcttcttcttcttcttcttcttcttcttcttcttcttcttcttcttcttcttcttcttcttcctcttcctcttctccttctccttctccttctccttctcctcctcctcctcctcctcctccttctcctcttcctcctcttcctcctcctcctcctcctcttcctcctccttcttcttcttctccttcttcttcttcttcttcttcttctggctctcaaacaccagttccttaaacatttaaacaaattttttgcctgcgtatatgtatgtgtgccatgtgtgtgcctggtgcctgcagtctagaagaggggtgttgggtcccctggagctggagttacagatggttgtgaggtattgtgtgggtgcttggaccggaacctgagttctcggcaagaacaagtgcttttaatcactgagccttctcttccagccccagaccagttctctggacgactcggagataagcatagcagtgcatacccagaatagcagcacttaggttgaggcaggaggattgtcattggtttgagaccaacctggattgctagttatagcttcaacacacacactcatgcacatgacttaggagcaggtgatatgactctaaagtaactaaaattgggtggaaatcacggctaggtttctattgactggaaaatgcttatatgtgaactccctccttctggtataagctatctattctagtctagaattccttaatggagatccccgtcatttactttttcccctgttacacctgatgagcctgatccaactgagggtcacagaacaagaacagtggcagtggggtggcagctggggggatttggaatgctctgcattcatctttggttagtccttgtgactttagcaagagctagctctgctcctgtcctctacttgggaggacatttagagatccactcccatgccctcgcctgcaggctcgcctagtttgtcccttgtgtgtgtggggtggggtgggggggactaactcccagcagattccctccctgcctaaccctccgccaagcccactcaggattccgtggtccaaggccagcccctgccccttgtggtgcagtaatgtgtgccaaagctctgctgaccaaaagaacaacagcagctagccctggataccctctctcccacccagcacctgtgccctcgatctttagctgtgggtgtcacgggccaagagcacctcgacagtgtaattttctggtggctggatacacagacaaaggtggggctacccctgagatccatggaggagtggaagggcggaagctatccagtcctgctgggggtggggaggagaagccagaggaccaagtggctctatcttctccatgaagatacctcagctggatggaatttgtctatatttagcaggtggctagcaggaggctgataagcagggctggggagggggcagtcctcataaatagtgagaacacaggacactgttcagtccctccttgggtggcctgcttgagctc

SEQ ID NO: 2 - DNA, 1100 bp, Mus sp.
cttttaatcactgagccttctcttccagccccagaccagttctctggacgactcggagataagcatagcagtgcatacccagaatagcagcacttaggttgaggcaggaggattgtcattggtttgagaccaacctggattgctagttatagcttcaacacacacactcatgcacatgacttaggagcaggtgatatgactctaaagtaactaaaattgggtggaaatcacggctaggtttctattgactggaaaatgcttatatgtgaactccctccttctggtataagctatctattctagtctagaattccttaatggagatccccgtcatttactttttcccctgttacacctgatgagcctgatccaactgagggtcacagaacaagaacagtggcagtggggtggcagctggggggatttggaatgctctgcattcatctttggttagtccttgtgactttagcaagagctagctctgctcctgtcctctacttgggaggacatttagagatccactcccatgccctcgcctgcaggctcgcctagtttgtcccttgtgtgtgtggggtggggtgggggggactaactcccagcagattccctccctgcctaaccctccgccaagcccactcaggattccgtggtccaaggccagcccctgccccttgtggtgcagtaatgtgtgccaaagctctgctgaccaaaagaacaacagcagctagccctggataccctctctcccacccagcacctgtgccctcgatctttagctgtgggtgtcacgggccaagagcacctcgacagtgtaattttctggtggctggatacacagacaaaggtggggctacccctgagatccatggaggagtggaagggcggaagctatccagtcctgctgggggtggggaggagaagccagaggaccaagtggctctatcttctccatgaagatacctcagctggatggaatttgtctatatttagcaggtggctagcaggaggctgataagcagggctggggagggggcagtcctcataaatagtgagaacacaggacactgttcagtccctccttgggtggcctgcttgagctc

(19) United States
(12) Patent Application Publication
(10) Pub. No.: US 2004/0175699 A1
(43) Pub. Date: Sep. 9, 2004

(54) Myocardium-specific promoter

(76) Inventors: Marc Fiszman, Paris (FR); Bernard Prudhon, Sartrouville (FR); Yves Fromes, Paris (FR)

Correspondence Address: Oblon Spivak McClelland, Maier & Neustadt, 1940 Duke Street, Alexandria, VA, 22314, US

(21) Appl. No.: 10/332,767

(22) PCT Filed: Jul. 12, 2001
(86) PCT No.: PCT/FR01/02258

(30) Foreign Application Priority Data
Jul. 12, 2000 (FR) 00/09090

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

Myocardium-specific promoter derived from the MYBPC gene of a mammal and comprising the regulatory cis-elements necessary and sufficient to direct the specific expression of a gene in the myocardium. Use for treating myocardial diseases.

The present invention relates to a promoter which is active in the myocardium and to the use thereof for expressing genes of interest in this tissue, in particular for treating myocardial diseases.

The modification of myocardial functions, of genetic or acquired origin, may induce serious pathological conditions, for example familial hypertrophic cardiomyopathy, myocardial infarction and cardiac rhythm disorders.

The possibility of modulating the responses of the cardiac muscle cells (cardiomyocytes) by directing the expression of a gene of interest in these cells may have many applications, such as for example:

in the case of cardiomyopathies of genetic origin, synthesizing the functional non-mutated cardiac protein, such as cardiac C-protein (familial hypertrophic cardiomyopathy),

in the case of acquired pathological conditions:

controlling cell division, either so as to stimulate it, for example in order to regenerate a functional muscle after an infarction, or so as to slow it down, for example in order to inhibit the growth of a cardiac tumour. This may be obtained, for example, by producing dominant negative mutants for growth factor receptors or by using antisense RNA,

controlling the contraction strength or the contraction rhythm of the cardiac muscle.

Furthermore, studying the functions in vivo of cardiomyocytes, and also modifying these functions for therapeutic purposes, requires using animal models for individually evaluating the role of each of the proteins expressed by cardiomyocytes, whether this involves overexpressing or underexpressing a protein produced naturally by these cells, for evaluating the activity of novel potentially therapeutic molecules, or for studying the functional consequences of the expression of a heterologous protein.

In order to be able to effectively use the potentialities of gene transfer into cardiac muscle, vectors must be available, which make it possible to obtain stable expression of a gene of interest in cardiomyocytes, in vitro and also in vivo, as must effective and reliable techniques for transferring therapeutic genes into the tissues concerned.

This expression must also, in certain cases, be specific for this tissue, in order to avoid the problems which may result from ubiquitous expression since certain viral vectors, such as adenoviruses, are capable of penetrating into many tissues.

At the current time, a certain number of proteins are known to be expressed more or less specifically in cardiac muscle, and their promoters constitute potential candidates for the tissue-specific expression of a heterologous gene; myosin light chains (MLCs) (O'BRIEN et al., Proc. Nat. Acad. Sci., 1993, 90, 5157-5161; LEE et al., J. Biol. Chem.,1992, 267, 15875-15885; KELLY et al., J. Cell Biol., 1995, 129, 383-396), the α-myosin heavy chain (α-MHC) (SUBRAMANIAM et al., J. Biol. Chem., 1993, 268, 4331-4336), the β-myosin heavy chain (β-MHC) (RINDT et al., J. Biol. Chem., 1993, 268, 5332-5338), cardiac troponin T (cTnT) (IANNELLO et al., J. Biol. Chem., 1991, 266, 3309-3313; CHRISTENSENJ et al., Mol. Cell. Biol., 1993, 13, 6752-6765), muscle creatinine kinase (MCK) (AMACHER et al., Mol. Cell. Biol., 1993, 13, 2753-2764), atrial natriuretic factor (ANF) (SEIDMAN et al., Can. J. Physiol. Pharmacol., 1991, 69, 1486-1492; ARGENTIN et al., Mol. Cell. Biol., 1994, 14, 777-790), α-cardiac actin (α-cA) (BIBEN et al., Dev. Biol., 1996, 173, 200-212), and the type A and type B natriuretic peptides (ANP and BNP, respectively) (DUROCHER et al., Dev. Genet., 1998, 22, 250-262).

Some of the promoters which regulate the expression of the genes above have been cloned and used in vivo to direct the expression of heterologous genes in transgenic mice, in particular those of the MLC2v (HUNTER et al., J. Biol. Chem., 1995, 270, 23173-23178), αCa (BIBEN et al., mentioned above), ANF (FIELD et al., Science, 1988, 239, 1029-1033), α- and β-MHC (RINDT et al., Transgenic Res., 1995, 4, 397-405; KNOTTS et al., Dev. Dyn., 1996, 206, 182-192) and cTnT (ZHU et al., Dev. Biol., 1995, 169, 487-503) genes.

Even though these sequences make it possible to express genes at the cardiac level, their expression profile is either restricted to a compartment of the heart or is not specific for the heart, at stages of embryonic development or in the adult (LYONS et al., J. Cell. Biol., 1990, 111, 2427-2436 and J. Cell. Bio., 1990, 111, 1465-1476).

By way of example:

the promoter of the β-MHC gene is not suitable for transgenic expression in the adult; its expression is restricted to the ventricles from the ninth day of development in mice, and drops a few days before birth (NG et al., Circ. Res., 1991, 68, 1742-1745),

the promoter of the MLC2v gene is restricted to the ventricles (HUNTER et al., mentioned above) (O'BRIEN et al., mentioned above),

the promoter of the ANF gene is restricted to the left ventricle during embryonic development before becoming specific for the atria in the adult (ZELLER et al., Genes Dev., 1987, 1, 693-698; FIELD et al., mentioned above),

the promoter of the α-MHC gene is expressed in the atria during embryonic development and its expression in the ventricles increases a few days before birth and persists after birth (PALERMO et al., Cell. Mol. Biol. Res., 1995, 41, 501-509; NG et al., mentioned above),

the promoter of the α-cA gene (GUNNING et al.; 1983, Mol. Cell. Biol., 1983, 3, 1985-1995) and the promoter of the cTnT gene (MAR et al., 1988, J. Cell. Biol., 1988, 107, 573-585; MAR et al., Symp. Soc. Exp. Biol., 1992, 46, 237-249) are expressed both in the myocardium and in skeletal muscle in the embryo and in the adult.

Studies carried out in various species have made it possible to isolate a certain number of transcription factors involved in cardiac differentiation and cardiogenesis. These factors bind to specific sequence motifs in the regulatory regions of these genes, named cis-elements for regulating transcription, and are capable of transactivating many cardiac genes expressed specifically in cardiomyocytes. Among these factors, the most well-known are: the MEF-2 (Myocyte Enhancer Factor-2) proteins, the homeobox factors Csx/Nkx2.5 and Mhox (Muscle Hox), the GATA factors, the ehand and dhand proteins and the TEF-1 (Transcription Enhancer Factor-1) factors (OLSON et al., Science, 1996, 272, 671-676; MABLY et al., Circ. Res., 1996, 79, 4-13).

However, although the participation of each of the factors above in regulating the expression of endogenous muscle genes is clearly established, the exact combination of these tissue-specific factors which is necessary and sufficient to express heterologous genes of therapeutic interest, specifically in the heart, has not been determined.

Added to this first degree of complexity in the control of tissue-specific expression, is an additional degree of complexity in so far as it has been reported that the particular combination of these tissue-specific factors with ubiquitous factors, such as factors related to SRF (Serum Response Factor), named RSRF (Related Serum Response Factor) (PARI et al., Mol. Cell. Biol., 1991, 11, 4796-4803; KARNS et al., J. Biol. Chem., 1995, 270, 480-487; PARKER et al., J. Biol. Chem. 1992, 267, 3343-3450; CHEN et al., Mol. Cell. Biol., 1996, 16, 6372-6384), Sp1 (SAFFER et al., 1991, Mol. Cell. Biol., 1991, 11, 2189-2199) and Egr-1 (MUTERO et al., J. Biol. Chem., 1995, 270, 1866-1872; THOMPSON et al., J. Biol. Chem., 1991, 266, 22678-22688; GUPTA et al., J. Biol. Chem., 1991, 266, 12813-12816; PARMACECK et al., Mol. Cell. Biol., 1994, 14, 1870-1885; ZHU et al., Mol. Cell. Biol., 1993, 13, 4432-4444), also influence the differential expression of muscle genes in the heart and may represent an additional mechanism for controlling the transcription of genes specifically in the heart (SARTORELLI et al., Circ. Res., 1993, 72, 925-931; LYONS et al., Curr. Opin. Genet. Dev., 1996, 6, 454-460; OLSON et al., mentioned above; MABLY et al., mentioned above; FIRULLI et al., Trends Genet., 1997, 13, 364-369; DUROCHER et al., 1998, mentioned above).

Consequently, no promoter has yet been characterized which allows the expression of a gene of interest in all the compartments of the heart, early in the embryo and in long-lasting fashion in the adult. In addition, the combination of cis-elements which is necessary and sufficient to ensure the expression of a gene specifically in cardiac muscle has not been identified.

The MYBPC3 cDNA and gene encoding human cardiac C-protein have been isolated (KASAHARA et al., J. Clin. Invest., 1995, 94, 1026-1036). The MYBPC3 gene is at least 21 kb in size, it is composed of 35 exons (CARRIER et al., Circ. Res., 1997, 80, 427-434) and it is located on band p11.2 of chromosome 11 (11p11.2) (GAUTEL et al., Circ. Res., 1998, 82, 124-129). However, no sequence which controls the transcription of this gene has been precisely identified.

Among the proteins which appear to be expressed specifically in the myocardium, C-protein is a protein which is abundant in striated muscles and which represents 2 to 4% of the fibrillar proteins (OFFER et al., J. Mol. Biol., 1973, 74, 653-676). It is part of a set of proteins having structural and/or regulatory functions associated with the myosin heavy chain in the thick filament (EPSTEIN et al., Science, 1991, 251, 1039-1044).

Three isoforms of C-protein exist, each one encoded by a gene and expressed specifically in a muscle type: rapid skeletal C-protein, slow skeletal C-protein and cardiac C-protein (cMyBPC) (YAMAMOTO et al., J. Biol. Chem., 1983, 258, 8395-8401).

The expression of cardiac C-protein is restricted exclusively to cardiac muscle and it is never found in skeletal muscle throughout development in humans and mice, and the transcripts of the MYBPC3 (Myosin Binding Protein-C 3-cardiac) gene encoding cardiac C-protein are present only in the heart, this being uniformly, in mouse or human embryos (GAUTEL et al., mentioned above; FOUGEROUSSE et al., Circ. Res., 1998, 82, 130-133).

In humans, the MYBPC3 gene encoding the cardiac isoform is one of the eight genes involved in cardiac disorders of the HCM type (TOWBIN et al., Curr. Opin. Cell. Biol., 1998, 10, 131-139; MOGENSEN et al., J. Clin. Invest., 1999, 103, 39-43). HCM is a familial pathological condition which is transmitted in Mendelian autosomal dominant way, characterized by unexplained hypertrophy of the left ventricle, predominant at the interventricular septum and associated with broad cellular and tissue disorganization (HENGSTENBERG et al., J. Mol. Cell. Cardiol., 1994, 26, 3-10).

Using a mouse genomic clone comprising a fragment of the MYBPC3 gene encoding cardiac C-protein, the inventors have now identified and cloned the sequence of the promoter of the MYBPC3 gene and have also located regulatory sequences necessary and sufficient for the tissue-specific activity of this promoter. These studies have enabled them to isolate fragments of this promoter which are capable of activating the expression of a gene specifically and exclusively in the myocardium at all stages of development, from the embryo to the adult.

A subject of the present invention is a nucleic acid molecule derived from a mammalian MYBPC gene, which comprises the regulatory cis-elements necessary and sufficient to direct the specific expression of a gene in the myocardium.

The nucleic acid molecule according to the invention essentially consists of the sequences necessary for controlling the initiation of transcription of the MYBPC3 gene (basal promoter), and also of the sequences involved in the cis-regulation of the initiation of transcription, this being via mechanisms similar to the natural mechanisms for regulating the transcription of the endogenous MYBPC3 gene.

The nucleic acid molecules in accordance with the invention may advantageously be used to direct the expression of a heterologous gene, in particular a gene of therapeutic interest.

For the purpose of the present invention, the term “heterologous” relative to a sequence of a given gene, is intended to mean any nucleic acid sequence other than those which, naturally, are immediately adjacent to said sequence.

Among the sequences necessary for controlling the initiation of transcription, mention may be made in particular of the canonical motifs recognized by RNA polymerase, such as the TATA box and CAT box sequences.

Among the potential sequences involved, in cis, in the regulation of transcription in cardiac muscle, mention may be made of the binding sequences for ubiquitous or cardiac muscle-specific transcription factors, such as the binding sequences for the factors described above.

Unexpectedly, the inventors have shown that a fragment of the MYBPC3 gene, comprising the TATA box and two GATA-4 sites, is capable of activating the expression of a heterologous gene only in cardiomyocytes in vitro and in vivo.

According to an advantageous embodiment of the invention, said nucleic acid molecule comprises two binding sequences for the GATA-4 transcription factor (canonical motif 5′-(A/G) GATA(A/G)-3′).

Advantageously, one of the GATA-4 sites is located between positions −60 and −70 or between positions −1010 and −1020, relative to the transcription initiation site of the MYBPC3 gene.

The GATA factors belong to a family of nuclear proteins which have a specific DNA-binding domain consisting of two adjacent zinc fingers of the C2/C2 family (WHYATT, EMBO J., 1993, 12, 4993-5005). The C-terminal zinc finger associated with an adjacent basic region constitutes the minimum domain for recognition of the canonical motif 5′-(A/G)GATA(A/G)-3′ (KO et al., Mol. Cell. Biol., 1993, 13, 4011-4022; MERIKA et al., Mol. Cell. Biol., 1993, 13, 3999-4010). Six GATA factors have been characterized in vertebrates, including the GATA-4/5/6 factors which are involved, firstly, in cardiogenesis and cardiac differentiation (JIANG et al., Dev. Biol., 1996, 174, 258-270; GOVE et al., EMBO J., 1997, 16, 355-368; GREPIN, Development, 1997, 124, 2387-2395), and, secondly, intestinal epithelial cell differentiation (GAO, Mol. Cell. Biol., 1998, 18, 2901-2911). The GATA-4/5/6 factors are capable of transactivating many cardiac genes expressed in cardiomyocytes (LAVERRIERE et al., J. Biol. Chem., 1994, 269, 23177-23184; KELLEY et al., Development, 1993, 118, 817-827; JIANG et al., mentioned above; MORRISEY et al., Dev. Biol., 1996, 177, 309-322; 1997, 183, 21-36; PARMACECK et al., Mol. Cell. Biol., 1992, 12, 1967-1976). For example, the GATA-4 factor activates the genes of the sarcomeric proteins, such as troponin C.

Advantageously, said nucleic acid molecule also comprises one or more binding site(s) for a factor involved in restricting the transcription of the MYBPC3 gene to cardiomyocytes.

Advantageously, said binding site(s) is (are) selected from the group consisting of: the binding sequences for the NFAT-3 factor, the binding sequences for Ets proteins and the binding sequences for RSRF factors.

The inventors have also observed that the sequence which extends from position −1500 to −800 relative to the transcription initiation site for the MYBPC3 gene is involved in restricting the transcription of the gene to cardiomyocytes.

The inventors have analysed said sequence and have located potential binding sites:

for the NFAT-3 (“Nuclear Factor of Activated T cells-3”; canonical motif 5′-(A/T)GGAAAAT-3′) factor,

for Ets proteins (Ets canonical motif 5′-GGA(A/T)-3′, and

RSRF factors (canonical motif of the CarG type: 5′-CC(A/T)GG-3′).

The NFAT-3 factor, which is capable of interacting with the GATA-4 factor, is expressed in the heart, it plays a role in cardiogenesis (DE LA POMPA et al., Nature, 1998, 392, 182-186; RANGER et al., Nature, 1998, 392, 186-190) and it is also involved in cardiac hypertrophy (MOLKENTIN et al., Cell, 1998, 93, 215-226).

The Ets proteins regulate tissue-specific expression by activation and repression mechanisms (CONRAD et al., Mol. Cell. Biol., 1994, 14, 1553-1565; ROSEN et al., J. Biol. Chem., 1994, 269, 15652-15660; UMEZAWA et al., Mol. Cell. Biol., 1997, 17, 4885-4894), among which, the ERP/Net protein, which is expressed in many tissues, is also present in the heart (LOPEZ et al., Mol. Cell. Biol., 1994, 14, 3292-3309; PRICE et al., EMBO J., 1995, 14, 2589-2601), and it would appear that it may interact with the binding site for RSRFs (PRICE et al., mentioned above; LOPEZ et al., mentioned above; WASYLYK et al., Eur. J. Biochem., 1993, 211, 7-18).

The binding of these factors to elements of this sequence may be responsible for restricting the transcription of the MYBPC3 gene to cardiomyocytes, and represents an additional mechanism for controlling the activity of the endogenous MYBPC3 promoter in cardiomyocytes.

Advantageously, the GATA-4 sites, and also the NFAT-3, Ets and RSRF sites, if they are present, are located, respectively, at the following positions, relative to the transcription initiation site:

GATA-4 site: −63/−58 and −1015/−1010,

NFAT-3 site: −822/−815, −850/−844,

Ets site: −935/−932, −1102/−1099, and

RSRF sites of the CarG type: −861/−853; −870/−865; −1231/−1221.

A nucleic acid molecule in accordance with the invention is represented, for example, by:

the sequence which extends from position −1535 to position +28 relative to the transcription initiation site of the murine MYBPC3 gene; this sequence is represented in the attached sequence listing under the number SEQ ID NO: 1, and

the sequence represented in the attached sequence listing under the number SEQ ID NO: 2, which extends from position 464 to position 1563 of the sequence SEQ ID NO: 1 above.

It should be clearly understood that the nucleic acid molecules specified above, which may be obtained from the murine MYBPC3 gene, constitute only an illustration of the subject of the invention. The latter also encompasses, in particular, nucleic acid molecules which reproduce homologous sequences existing in humans or in other mammals, and which may be obtained by those skilled in the art using conventional molecular biology techniques.

Similarly, using the nucleic acid molecules comprising at least the cis-elements specified above for regulating the MYBPC3 gene, those skilled in the art may construct various nucleic acid molecules in accordance with the invention, for example by mutating or deleting sequences located outside the regulatory cis-elements and/or, optionally, substituting them with other sequences.

The nucleic acid molecules comprising the cis-elements for regulating the MYBPC3 gene may be combined with regulatory cis-elements, or with a basal promoter, originating from genes other than the MYBPC3 gene, according to various combinations, so as to obtain chimeric nucleic acid molecules which differ from one another by their level of activity and their degree of specificity.

The nucleic acid molecules in accordance with the invention may be used to control the expression of a heterologous gene in mammalian cells and, advantageously, to obtain specific expression in cardiac muscle cells.

The subject of the present invention also encompasses the recombinant nucleic acid molecules comprising at least one nucleic acid molecule in accordance with the invention, linked to at least one heterologous sequence.

The subject of the present invention encompasses, in particular:

a) Expression Cassettes Comprising:

a nucleic acid molecule in accordance with the invention; it may be a sequence derived from the MYBPC3 gene, or a chimeric sequence, comprising at least the cis-elements for regulating the MYBPC3 gene as defined above,

and a heterologous sequence placed under the transcriptional control of said nucleic acid molecule;

b) recombinant vectors comprising an insert consisting of a nucleic acid molecule in accordance with the invention. Advantageously, these expression vectors comprise at least one expression cassette as defined above.

Many vectors into which a nucleic acid molecule of interest may be inserted in order to introduce it and to maintain it in a eukaryotic or prokaryotic host cell are, in themselves, known; the choice of a suitable vector depends on the use envisaged for this vector (for example replicating the sequence of interest, expressing this sequence, maintaining this sequence in extrachromosomal form or integrating it into the host's chromosomal material), and also on the nature of the host cell.

A subject of the invention is also prokaryotic or eukaryotic cells transformed with at least one nucleic acid molecule in accordance with the invention. Preferably, these cells are animal cells, in particular mammalian cells. They may be cardiac muscle cells or totipotent embryonic cells capable of differentiating into cardiac muscle cells.

Transformed cells in accordance with the invention may be obtained by any means, known in themselves, which make it possible to introduce a nucleic acid molecule into a host cell. For example, in the case of animal cells, use may be made, inter alia, of viral vectors, such as adenoviruses, retroviruses, lentiviruses and AAVs, into which the sequence of interest has been inserted beforehand; said sequence (isolated or inserted into a plasmid vector) may also be associated with a substance which allows it to cross the host-cell membrane, for example a preparation of liposomes, of lipids or of cationic polymers, or may be injected directly into the host cell, in the form of naked DNA.

The gene transfer into cardiac muscle may, for example, be carried out using recombinant adenoviruses, recombinant adenovirus-associated viruses (AAVs), liposomes, lipids or cationic polymers, or by direct injection of nucleic acid molecules.

These viral or nonviral vectors may be administered either by direct injection into the myocardium, by coronary infusion (BARR et al., Gene Therapy, 1994, 1, 51-58; BUDKER et al., Gene Therapy, 1998, 5, 272-276) or by injection into the pericardial envelope (LIM et al., Circulation, 1991, 83, 2007-2011; MUHIHAUSER et al., Gene Therapy, 1996, 3, 145-153; ROTHMANN et al., Gene Therapy, 1996, 3, 919-926).

The inventors have also obtained transgenic animals in which a heterologous gene is placed under the transcriptional control of a nucleic acid molecule comprising the cis-elements for regulating the MYBPC3 gene, according to the invention, and have thus noted that the properties of this nucleic acid molecule, and in particular the specificity of expression in cardiac muscle cells, manifest themselves not only ex vivo, but also in vivo at all the stages of development, from the embryo to the adult.

A subject of the present invention is animals, and in particular nonhuman transgenic mammals, characterized in that all or some of their cells are transformed with a nucleic acid molecule according to the invention. They are, for example, animals into which has been introduced a gene of interest under the control of cis-elements for regulating the MYBPC3 gene or of a chimeric promoter constructed from the cis-elements for regulating this gene, which confer the specific expression in cardiac muscle cells; the gene of interest is then expressed specifically in cardiac muscle cells.

The transformed cells and the transgenic animals in accordance with the invention can, in particular, be used as models for studying and/or modifying the expression of various genes in cardiac muscle cells.

The subject of the invention is also the use of nucleic acid molecules in accordance with the invention, for producing medicinal products, in particular medicinal products intended for the treatment of myocardial pathological conditions.

The present invention will be more clearly understood using the further description which follows, which refers to examples describing the production and characterization of the promoter of the MYBPC3 gene, and of the fragments thereof, which allow specific expression in cardiac muscle, and also to the use thereof for expressing heterologous genes specifically in cardiac muscle cells in cell cultures and in transgenic animals, and also to the attached drawings in which:

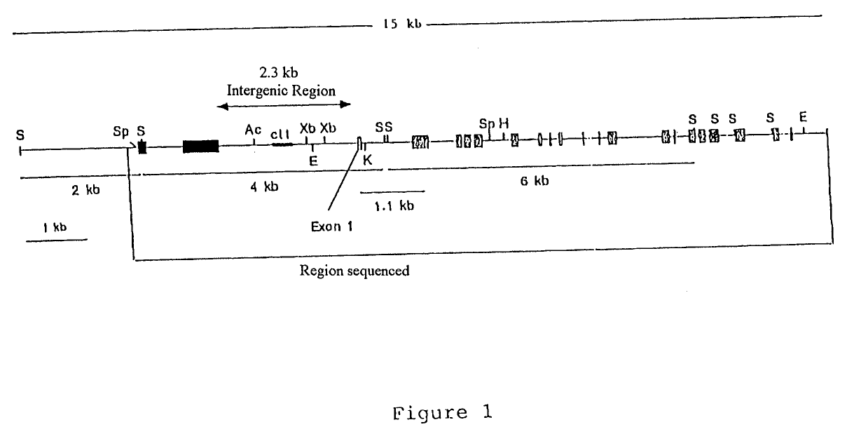

FIG. 1 illustrates the restriction map and the organization of the introns and exons of the 15 kb fragment of the MYBPC3 gene isolated from a mouse genomic library. This fragment comprises a 2.3 kb sequence between the Spi-1 gene and the MYBPC3 gene (intergene region), containing the promoter of the MYBPC3 gene. S: SacI; Sp: SphI; Ac: AccI; Xb: XbaI; H: HindIII; E: EcoRI; K: KpnI. The exons of the Spi-1 gene are represented in black and the exons of the MYBPC3 gene are represented by shading;

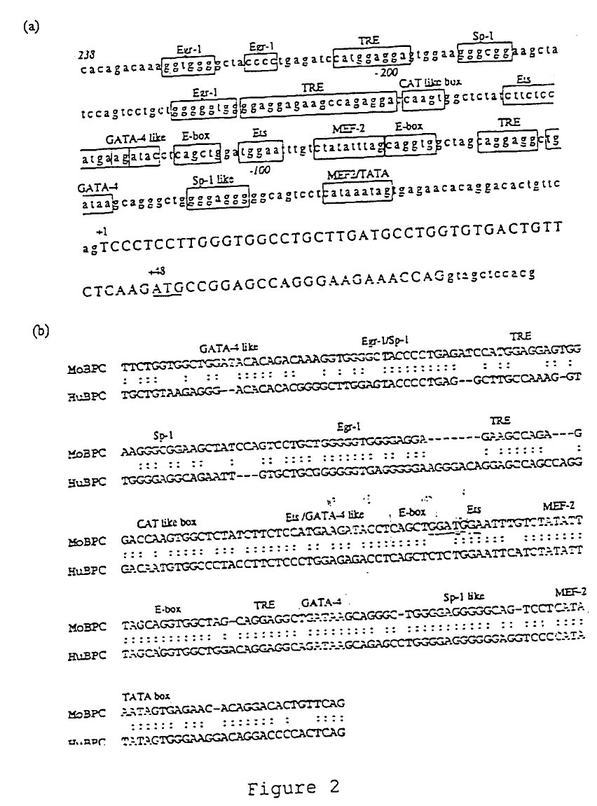

FIG. 2 illustrates the comparison of the proximal sequence of the promoter of the human and murine MYBPC3 gene. (a) The sequence which extends from position −238 to position +83, relative to the transcription initiation site (+1) of the mouse MYBPC3 gene is given and the potential binding sites for transcription factors are boxed. (b) The sequence which extends from position −255 to position +1, relative to the transcription initiation site, of the mouse MYBPC3 gene is aligned with the corresponding sequence of the human MYBPC3 gene (accession number Y10129) and the potential binding sites for transcription factors are boxed;

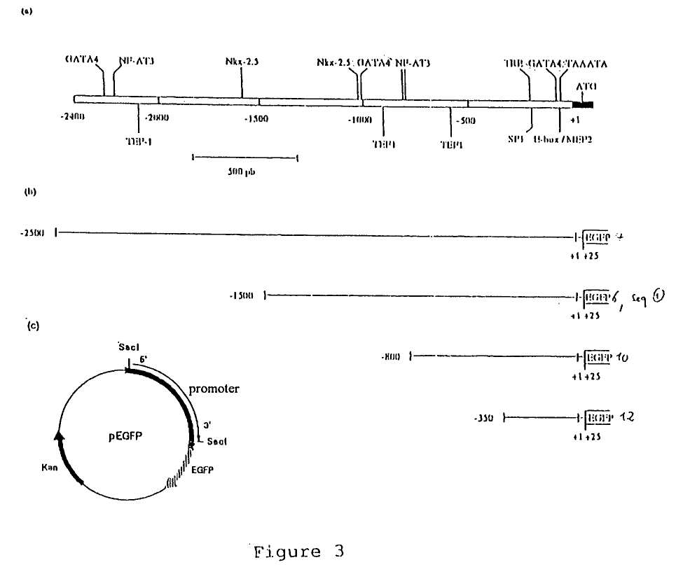

FIG. 3 illustrates the structure of the four fragments of, respectively, 2.5; 1.5; 0.8 and 0.35 kb of the promoter of the MYBPC3 gene, coupled to the EGFP reporter gene in the pEGFP vector;

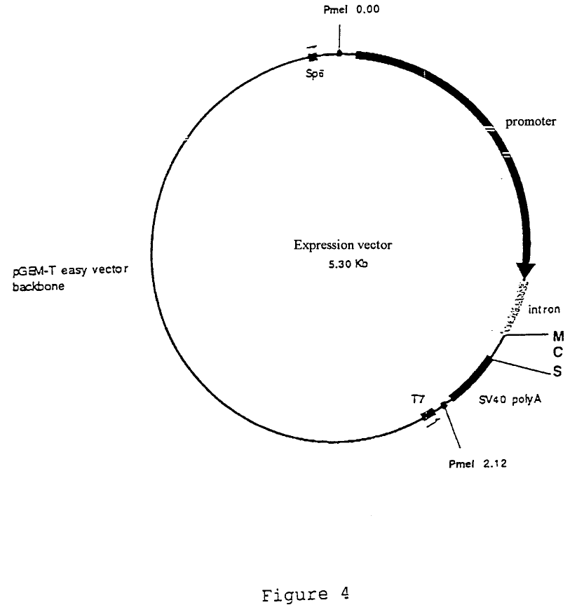

FIG. 4 illustrates the restriction map of an expression vector containing the 1.5 kb fragment of the promoter of the MYBPC3 gene.

### EXAMPLE 1

### Molecular Cloning and Chromosomal Location of the Promoter of the Murine MYPC3 Gene

A 15 kb fragment of the MYBPC3 gene was isolated by screening a λFIXII mouse genomic DNA library using a 215 bp probe corresponding to the 5′ end of the coding sequence of cardiac C-protein.

Three fragments, of 2 kb, 4 kb and 6 kb, obtained by restriction of the 15 kb fragment derived from the genomic clone with the SacI enzyme were subcloned at the SacI site of the pBluescript plasmid (STRATAGENE). The plasmids obtained, named pSac2, pSac4 and pSac6, respectively, were sequenced and the sequence of the above fragments was compared with the databanks.

The restriction map of the 15 kb fragment is represented in FIG. 1; it contains exons 1 to 20 of the gene of cardiac C-protein, the last two exons of the Spi-1 oncogene (accession number: X17463) and 2.3 kb of intergene sequence.

In humans, the chromosomal organization of the Spi-1 (MOREAU-GACHELIN, Oncogene, 1989, 4, 1449-1456) and MyBPC3 (GAUTEL et al., mentioned above) genes is similar to that observed in mice; they are both located in the p11.2 region of chromosome 11.

The 2.3 kb intergene region comprising the promoter of the MYBPC3 gene, derived from the pSac4 plasmid, was then analysed.

### EXAMPLE 2

### Analysis of the Promoter of the Human and Murine MYBPC3 Gene

2.1. Determination of the Transcription Initiation Site of the Murine MYBPC3 Gene

The transcription initiation site was determined by the primer extension technique and by cloning the 5′ end of the messenger RNA of cardiac C-protein, using, respectively, the AMV Reverse Transcriptase Primer Extension System kit (PROMEGA) and the 5′RACE (Rapid Amplification System of cDNA ends) kit, according to the manufacturer's instructions. The results show that the transcription initiation site of the MYBPC3 gene is located 47 bp upstream of the ATG.

2.2. Search for Specific Binding Motifs for Transcription Factors in the Human and Murine MYBPC3 Genes

The alignment of the 2.3 kb sequence with the sequence of the human gene (accession number Y10129) shows close to 80% homology between positions −200 bp and +1 (FIG. 2b).

The 2.3 kb sequence was analysed using the MatInspector program, which makes it possible to search for attachment sites for factors involved in transcription (QUANDT et al., Nucleic Acid Res., 1995, 23, 4878-4884; http://www.gsf.de/biodv/matinspector.html). Sequence elements described as being important in the regulation of genes encoding muscle proteins were also sought directly on these sequences.

The potential binding sites for ubiquitous or heart-specific transcription factors identified in the proximal sequence −238 to +83 of the murine promoter, and the conservation of these sequences in the human promoter, are given on FIGS. 2a and 2b, respectively.

The analysis of the murine promoter shows that the transcription initiation site is located 30 bp downstream of the first nucleotide of a TATA box (5′-TAAATA-3′) recognized by RNA polymerase II, which is also present in the human promoter. In addition, no canonical motif of the CAAT type (CAT box) was identified in the first 200 nucleotides upstream of the transcription initiation site of the murine or human promoter. However, it is possible that the CAAGT sequence of the murine promoter or the CAAT sequence of the human promoter, located between −137/−141 bp, may play the role of a CAT box.

Binding sites for transcription factors, below, were identified in the murine promoter at the positions specified; those identical in the human promoter are indicated in bold:

GC-rich binding sites for the Sp1 and Sp1-like factors:

−47/−41; −192/−187; −282/−278

GC-rich binding sites for the Egr-1 factors:

−169/−161; −227/−222; −293/−287; −503/−490; −669/−662

AT-rich binding sites for the MEF-2 factors:

−32/−24; −92/−83

The first binding site for MEF-2 covers the TATA box and the second is adjacent to another cis-element for regulating transcription (E box).

AT-rich binding sites for homeodomain proteins such as MHox:

−260/−252; −758/−752; −813/−806; −1071/−1063; −1710/−1697

GATA-4 canonical motifs (5′-(A/G)GATA(A/G)-3′):

−63/−58; −1015/−1010; −2239/−2234

GATA-4-like motif:

−117/−112; −244/−239

Nkx2.5 canonical motif (5′-TNAAGTG-3′):

−981/−975; −1078/−1072; −1631/−1625 (on the antisense strand)

GTIIC, SphI and SpHII binding sites for the TEF-1 factors:

−105/−100; −640/−646; −812/−804; −2132/−2124.

The sites at position −105/−100 and −640/−646 are in juxtaposition to an E box.

Thyroid hormone-binding site (TRE: Thyroid Responsive Element):

−70/−64; −161/−143; −205/−198; −967/−959; −1592/−1585; −1611/−1604

Canonical motif 5′-CA(G/C)(C/G)TG-3′ (E box):

−82/−77; −109/−104

Canonical motif (5′-GGA(A/T)-3′) for the Ets factors:

−101/−95; −935/−932; −1102/−1099

Palindromic motifs with high affinity for the Ets factors:

−116/−128

AT-rich binding sites, of the CarG or GArC type, for the RSFR [sic] factors:

−820/−814; −848/−843; −870/−865; −861/−863; −1231/—1221; −1721/—1710

Canonical binding motif (5′-(A/T)GGAAAAT-3′) for the NFAT-3 (Nuclear Factor of Activated cells) factor:

−822/−815; −850/−844; −2200/−2193.

These results show that, firstly, many transcription factors are capable of binding to the promoter of murine and human MYBPC3 genes and, secondly, that most of the binding sites for these factors are conserved between the human and murine promoters.

However, the presence of these sites, even if they are canonical, does not mean, however, that they are directly involved in regulating the endogenous promoter. Consequently, only the functional analysis of the role of these various sites in the activity of the promoter of the MYBPC3 gene makes it possible to determine the minimum combination of these sequences which is capable of ensuring the tissue-specific expression of the MYBPC3 gene in the heart.

### EXAMPLE 3

### Determination of a Minimum Promoter Fragment Specific for Cardiac Cells

3.1 Promoter Activity of the 2.3 kb Intergene Sequence and of Fragments Derived from this Sequence

The intergene region was amplified by PCR using the pSac4 plasmid, so as to generate fragments of, respectively, 2.5 kb, 1.5 kb (SEQ ID NO: 1), 1.1 kb (SEQ ID NO: 2), 0.8 kb and 0.35 kb, having an identical 3′ end corresponding to position +28 relative to the transcription initiation site and a variant 5′ end (FIG. 3). In the 3′ primer, the ATG at position +24 to +26 of the MYBPC3 gene, which may disturb translation initiation at the first ATG of a heterologous gene cloned behind these sequences, is replaced with a SacI site. The amplification products obtained are cloned at the SacI site of the “pGEM-T easy vector” plasmid (PROMEGA) and then subcloned in both orientations (sense and antisense) at the SacI site of the pEGFP-1 plasmid (CLONTECH) which contains the EGFP (Eukaryotic Green Fluorescent Protein) cDNA. The plasmids obtained are named pEGFP4 (2.5 kb), pEGF 6 (1.5 kb), pEGFP8 (1.1 kb), pEGF 10 (0.8 kb) and pEGFP12 (0.35 kb). The plasmids containing the insert in its antisense orientation are used as negative controls and the plasmid containing the 2.5 kb fragment is used as a positive control.

The plasmids containing the various promoter fragments are electroporated into embryonic carcinoma cells of mice of the C3H/He strain (P19 cells; Mc BURNEY et al., Nature, 1982, 299, 165-167). Stable transfectants which have integrated copies of these plasmids are selected in the presence of geneticin, and then maintained in the undifferentiated state or induced into differentiation.

P19 cells are totipotent embryonic cells capable of differentiating into cardiac cells in the presence of dimethyl sulphoxide at the final concentration of 1% v/v. Embroid bodies consisting of cardiac cells in contact with one another can be recognized by rhythmic beating areas, a characteristic phenotype of the developing heart in vivo (DOETSCHMAN, J. Embryol. Exp. Morphol., 1985, 87, 27-45; RUDNIKI, Dev. Biol., 1990, 138, 348-358; MALTSEV, Mech. Dev., 1993, 44, 41-50).

The P19 cells are maintained in the undifferentiated state in proliferation medium (DMEM (GIBCO), 15% foetal calf serum, 0.1 mM β-mercaptoethanol). After trypsinization, 2×10⁶ to 5×10⁶ cells in suspension are electroporated (GENE PULSER, BIO-RAD, set at 250 V and 500 μF) for 5 to 7 ms in a volume of 800 μl of phosphate buffer, pH 7.4, containing 25 μg of plasmid. The cells are then distributed into culture dishes containing 0.1% gelatin, supplemented with proliferation medium. 2 days after electroporation, the selection is carried out in proliferation medium containing 0.7 mg/ml of geneticin.

The differentiation is carried out in DMEM medium containing 20% of foetal calf serum and 1% of DMSO, using the hanging drop technique; the cells in suspension (25 000 cells/cm³) are deposited in 20 μl drops (500 cells) onto the lid of a cell culture dish and incubated for 3 days (these cells aggregate to form embroid bodies), and the embroid bodies are then detached and cultured in suspension for 4 days and, on the seventh day of differentiation they are transferred into a gelatinized culture medium allowing them to adhere to the support. The beating areas appear between the fourteenth and twentieth day of differentiation.

The EGFP expression is determined using a set of 15 embroid bodies observed with an inverted fluorescence microscope (OLYMPUS IX50/IX70+IX=FLA) and evaluated by the percentage of embroid bodies emitting fluorescence in the beating areas.

The results of this experiment are as follows:

1) No EGFP expression is observed in the differentiated or undifferentiated P19 cells electroporated with the plasmids in their antisense version, which demonstrates that the fluorescence observed is specific for the activity of the promoter.

2) With the pEGFP10 or pEGFP12 plasmid, very weak EGFP expression is observed in the undifferentiated P19 cells, and, in the P19 cells differentiated into cardiac cells, major EGFP expression is observed in the beating areas, with weak and variable expression in cells outside these beating areas.

3) With the pEGFP4, pEGFP6 and pEGFP8 plasmids, an absence of EGFP expression is observed in the undifferentiated P19 cells and EGFP expression is observed only in the beating areas in the P19 cells differentiated into cardiac cells.

These results make it possible to establish that only the 2.5 kb, 1.5 kb and 1.1 kb fragments contain all of the promoter sequences of the murine MYBPC3 gene which allow cardiac muscle cell-specific expression.

3.2. Determination of the Sequence Elements Involved in the Cardiac Specificity of the Promoter.

3.2.1. Mutagenesis of the GATA-4 Sites

It was previously shown (Example 2) that the promoter of the MYBPC3 gene contains 2 canonical GATA-4 motifs, the most proximal of which located between −63/−58 bp (GATA.1 site) is included in the 1.5 kb, 0.8 kb and 0.35 kb fragments, and the most distal of which (GATA.2 site) located between −1015/−1010 bp is included only in the 1.5 kb fragment (FIG. 3).

Since these motifs are important in the specific expression of cardiac genes, the TGATAA (GATA.1 site) and AGATAA (GATA.2 site) sequences were mutated independently into the XbaI restriction site (TCTAGA) in the pEGFP6 plasmid. The GATA.1 site present in the pEGFP10 and pEGFP12 plasmids was also mutated into the XbaI site. The plasmids obtained comprising these mutations are named pEGFP6.1, pEGFP6.2, pEGFP6.1.2, pEGFP10.1 and pEGFP12.1.

3.2.2 Transfection of the Mutated Plasmids into Non-Cardiac Cells

In order to verify that the mutations indeed prevent the attachment of the GATA-4 transcription factor, the pEGFP6 and doubly mutated pEGFP6 (pEGFP6.1.2) constructs are transiently transfected into non-cardiac cells (QT6 quail fibroblast line; MOSCOVICI et al., Cell, 1977, 11, 95-103), in the presence or absence of a plasmid vector for expressing the GATA-4 factor.

500 000 QT6 cells cultured in DMEM medium containing 10% of foetal calf serum are transfected with a mixture containing 1 μg of mutated or non-mutated pEGFP6 plasmid, 1 μg of the GATA-4 plasmid and 3 μl of non-liposomal lipid Fugene6 (BOEHRINGER), according to the manufacturer's recommendations. The cells transfected with the pEGFP6 and mutated pEGFP6 plasmids alone are used as a negative control. 48 hours after transfection, the fluorescence emission is observed using an inverted fluorescence microscope.

It is observed that only the construct mutated on the two sites is not transactivated by the expression of the GATA-4 factor. The other constructs are transactivated, to lesser or greater degrees, the greatest transactivation being obtained with the non-mutated pEGFP6 plasmid. These results therefore establish that only the two canonical sites at positions −63/−58 and −1015/−1010 allow good transactivation of the 1.5 kb promoter fragment by the GATA-4 factor.

3.2.3 Transfection of the Mutated Plasmids into Cardiac Cells

The mutated or non-mutated plasmids are electroporated into P19 cells differentiated into cardiac cells as described in Example 3.1.

The results of 2 independent series of experiments are given in Table I below.

TABLE I

| | | | Beating | Beating |
|---|---|---|---|---|
| | Mutated GATA-4 sites | | areas | areas |
| | GATA.1 | GATA.2 | expressing | expressing |
| | position | position | EGFP (%) | EGFP (%) |
| Plasmid | −63/−58 | −1015/−1010 | Experiment 1 | Experiment 2 |
| pEGFP 6 | − | − | 100 | 100 |
| pEGFP | + | − | 0 | 50 |
| 6.1 | | | | |
| pEGFP | − | + | 80 | 63 |
| 6.2 | | | | |
| pEGFP | + | + | 0 | 0 |
| 6.1.2 | | | | |
| pEGFP 12 | − | − | 93 | ND |
| pEGFP | + | − | 58 | ND |
| 12.1 | | | | |
| pEGFP 10 | − | − | ND | 80 |
| pEGFP | + | − | ND | 60 |
| 10.1 | | | | |

*ND = not determined

The results observed make it possible to establish that, in the cardiac cells:

1) the 1.5 kb fragment mutated on the two GATA sites exhibits no activity

2) the 1.5 kb fragment mutated on one or other of the GATA sites exhibits activities weaker than that of the non-mutated fragment

3) the shorter fragments (0.8 kb or 0.5 kb) which have none of the GATA sites, through deletion and/or mutation, still conserve an activity.

The results confirm that the 1.5 kb fragment constitutes a strong minimum promoter of the MYBPC3 gene, which is the most correctly regulated in cardiac cells, and makes it possible to establish that the two GATA-4 sites (−63/−58 and −1015/−1010) are essential for the activity of this promoter.

In addition, the results observed with the pEGFP10.1 and pEGFP12.1 plasmids compared to those observed for the pEGFP6.1.2 plasmid, make it possible to establish that a negative regulatory mechanism exists involving the −1500/−800 sequences.

The analysis of the 700 bp (see Example 3) reveals consensus binding motifs for the NFAT3 transcription factor [−822/−815 bp (5′-TGGAAAAT-3′), −850/−844 bp (5′-TGGAAAT-3′)].

In addition, the Ets sites (−935/−932, −1102/−1099) and the SRFR sites (−1231/−1221; −861/−853; −870/−865) are potential targets for the ERP/Net factor, which is expressed in the heart.

The NFAT-3 factor which binds to the canonical motif 5′-(A/T)GGAAAAT-3′ (HOEY et al., Immunity, 1995, 2, 461-472) is part of a multigene family, the members of which are expressed essentially in lymphocytes (RAO et al., Annu. Rev. Immunol., 1997, 15, 707-747). NFAT-3 is expressed in the heart and plays a role in cardiogenesis (DE LA POMPA et al., Nature, 1998, 392, 182-186; RANGER et al., Nature, 1998, 392, 186-190), it has also been reported that NFAT-3 interacts with GATA-4 so as to activate transcription of the BNP (Natriuretic Peptide type B) gene, which is involved in cardiac hypertrophy (MOLKENTIN et al., mentioned above).

The Ets proteins which recognize the sequence 5′-GGA(A/T)-3′ (TREISMAN et al., Curr. Opin. Dev. Genet., 1994, 4, 96-101) regulate tissue-specific expression via activation and repression mechanisms (CONRAD et al., Mol. Cell. Biol., 1994, 14, 1553-1565; ROSEN et al., J. Biol. Chem., 1994, 269, 15652-15660; UMEZAWA et al., Mol. Cell. Biol., 1997, 17, 4885-4894). Among these, the ERP/Net protein, which is expressed in many tissues, including the heart (LOPEZ et al., Mol. Cell. Biol., 1994, 14-3292-3309; PRICE et al., EMBO J., 1995, 14, 2589-2601), interacts with a single Ets binding site (LOPEZ et al., mentioned above), and it would appear that it may also interact with the binding site for SRFRs [sic] (PRICE et al., EMBO J., 1995, 14, 2589-2601; LOPEZ et al., mentioned above; WASYLYK et al., Eur. J. Biochem., 1993, 211, 7-18).

This negative regulatory mechanism represents an additional method for controlling specific expression in the heart for the 1.5 kb fragment of the promoter of the MYBPC3 gene.

### EXAMPLE 4

### Construction of an Expression Vector According to the Invention

An expression vector, named pU523, which allows the expression of a heterologous gene under the transcriptional control of the 1.5 kb fragment of the promoter of the MYBPC3 gene was constructed according to the following steps:

1) the SalI-NotI fragment of the multiple cloning site of the pSL1190 “superlinker phagemid” plasmid (PHARMACIA) was inserted between the SalI and NotI sites of the pEGFP-1 plasmid (CLONTECH), so as to give the pΔEGFP plasmid, deleted of the promoter and of the coding portion of the EGFP cDNA,

2) the XhoI-SmaI fragment of the pCMVβ plasmid (CLONTECH) containing an intron and also a splice donor site and a splice acceptor site (SD/SA) was inserted between the SalI and SmaI sites of the pΔEGFP plasmid,

3) a PmeI site was introduced by PCR at the ends of the Eco47III-Afl-II fragment of the pΔEGFP plasmid (700 bp), containing the intron, the SD/SA and the SV40 polyadenylation sequence,

4) the amplification product obtained was cloned into the pGEM-T easy vector plasmid (PROMEGA), so as to give the pPmeI plasmid, and

5) the SalI-HindIII fragment of the pGEM-T6 plasmid containing the 1.5 kb fragment of the promoter of the MYBPC3 gene, obtained as described in Example 3, was inserted between the XhoI and HindIII sites of the PmeI fragment of the pPmeI plasmid, to give the pU523 plasmid.

The map of the pU523 vector is illustrated by FIG. 4: it comprises the 1.5 kb fragment of the promoter of the MYBPC3 gene, a small intron, a multiple cloning site and a polyadenylation sequence, bordered by a rare restriction site (PmeI site) which makes it possible to isolate the expression cassette and to clone it into another vector.

### EXAMPLE 5

### Activity of the 1.5 kb Minimum Promoter in vivo in Transgenic Mice

The pEGFP6 plasmid, obtained as described in Example 3, was digested with the SacI and AflII endonucleases, and the restriction fragment obtained, which comprises the 1.5 kb fragment of the promoter of the MYBPC3 gene, linked to the EGFP gene, was purified. This fragment was injected into fertilized mouse eggs which were then reimplanted into pseudopregnant mice.

The EGFP expression in the 10 mouse lines obtained was analysed by direct observation of various organs using an inverted fluorescence microscope.

7 of the 10 lines strongly express the EGFP; this expression is detected only in the heart.

These results make it possible to affirm that the 1.5 kb fragment is active in vivo and makes it possible to express a heterologous gene at high levels, exclusively in the heart. The expression cassette as defined above therefore makes it possible to evaluate the activity of novel therapeutic molecules for cardiac or cardiovascular purposes, and to study the functional consequences of the expression of a heterologous protein in cardiac muscle.

### EXAMPLE 6

### Activity of the 1.5 kb Minimum Promoter in vivo in Cardiac Muscle after Injection of a Recombinant Vector Containing the Promoter Linked to the β-Galactosidase Reporter Gene

6.1. Injection of a Recombinant Plasmid

A recombinant plasmid, named pShMYBPC3-LacZ, containing the coding sequence of the lacZ gene, under the control of the 1.5 kb fragment of the promoter of the murine MYBPC3 gene, was constructed according to the following steps. The XhoI-SalI fragment of the pCMVβ plasmid (CLONTECH) containing the coding sequence of the lacZ gene was inserted at the SalI site of the pAdeasy adenovirus shuttle plasmid (HE et al., Proc. Nat. Acad. Sci., 1998, 95, 2509-2514). The plasmid obtained contains the coding sequence of the lacZ gene, flanked in 5′ by a splice acceptor site, by an intron and by a splice donor site, originating from the SV40 late region, and in 3′ by a polyadenylation site. This plasmid was then digested with the XhoI and HindIII endonucleases, and the XhoI-HindIII fragment of the pEGFP6 plasmid containing the promoter of the MYBPC3 gene was inserted between these two sites, so as to give the pShMYBPC3-LacZ plasmid.

Adult male Wistar rats were injected with 50 to 500 μg of the recombinant plasmid obtained, containing the β-galactosidase gene under the transcriptional control of the 1.5 kb fragment of the promoter of the MYBPC gene. 2 series of experiments were carried out on batches of 3 animals:

1) Intramyocardial injection, into the wall of the left ventricle, of 50 to 250 μg of recombinant plasmid in solute containing glucose at 5% W/V in an injection volume of 100 μl,

2) Intramuscular injection, into the Tibialis anterior skeletal muscle, of 50 to 500 μg of recombinant plasmid in solute containing glucose at 5% W/V in an injection volume of 100 μl.

The plasmid not containing a promoter was used as a negative control and the pCMVβ plasmid (CLONTECH) was used as a positive control for the expression of β-galactosidase in the two types of muscle tissue.

7 days after injection, the animals were sacrificed and the heart and muscle were removed. Transverse or longitudinal serial cryosections of these two organs were prepared.

After incubation in the presence of the substrate X-gal (5-bromo-4-chloro-3-indolyl-β-galactoside), the sections were observed under a microscope in order to analyse the cells expressing the β-galactosidase.

After injection of the control plasmid without promoter into the myocardium or into the skeletal muscle, no cell expressing β-galactosidase is detected in the two types of muscle.

After injection into the myocardium, many cells expressing β-galactosidase are detected, both in the animals having received the pCMVβ plasmid and in the animals having received the pShMYBPC3-LacZ plasmid.

On the other hand, after injection into the skeletal muscle, no cell expressing β-galactosidase is detected in the animals having received the pShMYBPC3-LacZ plasmid, whereas many cells expressing β-galactosidase are detected in the animals having received the pCMVβ plasmid.

These results make it possible to affirm that the in vivo administration of a plasmid expression vector containing a heterologous gene under the control of the 1.5 kb fragment of the MYBPC3 promoter makes it possible to express this heterologous gene only in the myocardium.

6.2 Injection of a Recombinant Adenovirus

A recombinant adenovirus, named AdMYBPC3-βgal, containing the β-galactosidase gene under the transcriptional control of the 1.5 kb fragment of the promoter of the MYBPC3 gene, in place of the E1 region of a ΔE1 defective adenovirus, was obtained by homologous recombination using the pShMYBPC3 adenovirus shuttle plasmid.

The recombinant adenovirus described by DAVIDSON et al. (Nature Genetics, 1993, 3, 219-223), containing the β-galactosidase gene under the transcriptional control of the ubiquitous cytomegalovirus promoter (AdCMV-βgal), was used as a control.

Batches of 3 adult male Wistar rats were injected with 10⁹ pfu (plaque-forming units) of the AdMYBP3-βgal or AdCMV-βgal adenovirus. 4 series of experiments were carried out:

1) intramyocardial injection, into the wall of the left ventricle, of the preparation of adenovirus in Ringer-lactate solute in an injection volume of 100 μl,

2) intramuscular injection, into the Tibialis anterior skeletal muscle, of the preparation of adenovirus in Ringer-lactate solute in an injection volume of 100 μl,

3) intrapericardial injection, according to the method described in FROMES et al. (Gene Therapy, 1999, 6, 683-688), of the preparation of adenovirus in Ringer-lactate solute in an injection volume of 750 μl,

4) intra-arterial injection of the preparation of adenovirus in Ringer-lactate solute in an injection volume of 100 μl.

7 days after injection, the animals were sacrificed and the heart, the muscle and the other organs (lung, diaphragm, liver, kidney, spleen) were removed. Transverse and/or longitudinal serial cryosections of these organs were prepared.

After injection into the skeletal muscle, no cell expressing the β-galactosidase is detected in the animals having received the AdMYBPC3-βgal, whereas many positive cells are observed in the animals having received the AdCMV-βgal.

After intrapericardial injection, the β-galactosidase expression is limited to the cells of the myocardium and also to cells of the spleen in the animals having received the AdMYBPC3-βgal, whereas cells expressing the β-galactosidase are observed in many organs (liver, heart, spleen, lung, kidney) of the animals having received the AdCMV-βgal.

After intra-arterial injection, no cell expressing the β-galactosidase is observed in the animals having received the AdMYBPC3-βgal, whereas cells expressing the β-galactosidase are observed in many organs, in particular in the liver, but never in the heart, of the animals having received the AdCMV-βgal.

These results make it possible to affirm that the in vivo administration, directly into the heart or systemically, of a recombinant adenovirus containing a heterologous gene under the control of the 1.5 kb fragment of the MYBPC3 promoter makes it possible to express this gene, at high levels, only in the myocardium.

This recombinant vector therefore makes it possible to limit the undesirable effects related to the expression of a heterologous gene in a tissue other than the myocardium.

1. Nucleic acid molecule capable of directing the specific expression of a gene in the myocardium, characterized in that it comprises the cis-elements for regulating transcription of the promoter of a mammalian MYBPC3 gene, which elements for regulating transcription comprise at least two binding sequences for a GATA-4 transcription factor.

2. Nucleic acid molecule according to claim 1, characterized in that it comprises a GATA-4 site between positions −60 and −70 or between positions −1010 and −1020, relative to the transcription initiation site.

3. Nucleic acid molecule according to claim 2, characterized in that it also comprises one or more binding sites for a transcription factor involved in restricting the transcription of the MYBPC3 gene to cardiomyocytes, said binding site(s) being selected from the binding sequences for the NFAT-3 factor, the binding sequences for Ets proteins and the binding sequences for RSRF factors.

4. Nucleic acid molecule according to claim 3, characterized in that said binding sites are located at the following position relative to the transcription initiation site:
GATA-4 sites: −63/−58; −1015/−1010;
NFAT-3 sites: −822/−815; −850/−844;
Ets sites: −935/−932; −1102/−1099;
RSRF sites of the CarG type: −861/−853; −870/−865; −1231/−1221.

5. Nucleic acid molecule according to any one of claims 1 to 4, characterized in that its sequence is selected from the group consisting of:
the sequence represented in the attached sequence listing under the number SEQ ID NO: 1, and
the sequence represented in the attached sequence listing under the number SEQ ID NO: 2.

6. Recombinant vector comprising an insert consisting of a nucleic acid molecule according to any one of claims 1 to 5.

7. Cell transformed with at least one nucleic acid molecule according to any one of claims 1 to 5.

8. Transformed cell according to claim 7, characterized in that said cell is a mammalian cell.

9. Nonhuman transgenic animal, characterized in that all or some of its cells are transformed with a nucleic acid molecule according to any one of claims 1 to 5.

10. Transgenic animal according to claim 9, characterized in that it is a mammal.

11. Use of a nucleic acid molecule according to any one of claims 1 to 5, for producing medicinal products.
